(19) United States
(12) Patent Application Publication
(10) Pub. No.: US 2023/0149223 A1
(43) Pub. Date: May 18, 2023

(54) WASTE COLLECTION DEVICE

(71) Applicant: Judith Snyder, Roseville, MN (US)

(72) Inventor: Judith Snyder, Roseville, MN (US)

(21) Appl. No.: 17/529,627

(22) Filed: Nov. 18, 2021

(51) Int. Cl. A61F 13/47; A61F 13/511; A61F 13/533

(52) U.S. Cl. CPC A61F 13/47; A61F 13/51108; A61F 13/533; A61F 2013/15121

(57) ABSTRACT

A waste collection device to connect to an interior of an undergarment, the waste collection device including a top layer, including an absorbent surface to absorb at least one liquid thereon, a plurality of reservoirs disposed within at least a portion of the absorbent surface to collect the at least one liquid therein, and a collection channel longitudinally disposed along at least a portion of the absorbent surface to collect at least one fecal matter therein, and a bottom layer removably connected to at least a portion of the top layer to prevent movement of at least one of the at least one liquid and the at least one fecal matter therethrough.

### BACKGROUND

### 1. Field

The present general inventive concept relates generally to waste collection, and particularly, to a waste collection device.

### 2. Description of the Related Art

At any given moment, a person may have an urge to use a restroom facility due to a need to urinate and/or defecate. However, restroom facilities are not always readily available, which is problematic for people when the urge to use the restroom facility is heightened. As such, any bowel movement will lead to an unwanted release of gas and/or fecal matter. Although a sudden discharge of waste is unpredictable, it can be embarrassing, messy, and inconvenient for the person to be able to reach the restroom facility in a timely manner.

Moreover, while there are diapers that can be used to collect waste, they are large and have a size similar to an undergarment. In other words, wearing a diaper adds another layer of clothing under the undergarment before shorts, pants, and/or skirts are worn.

Therefore, there is a need for a waste collection device that can protect the undergarment as well as effectively collect any sudden discharge of waste.

### SUMMARY

The present general inventive concept provides a waste collection device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a waste collection device to connect to an interior of an undergarment, the waste collection device including a top layer, including an absorbent surface to absorb at least one liquid thereon, a plurality of reservoirs disposed within at least a portion of the absorbent surface to collect the at least one liquid therein, and a collection channel longitudinally disposed along at least a portion of the absorbent surface to collect at least one fecal matter therein, and a bottom layer removably connected to at least a portion of the top layer to prevent movement of at least one of the at least one liquid and the at least one fecal matter therethrough.

The absorbent surface may have a zig zag pattern to increase a volume of absorbent material disposed thereon.

Each of the plurality of reservoirs may form a groove that is recessed with respect to a surrounding portion of the absorbent surface.

The collection channel may have less volume of absorbent material with respect to a surrounding portion of the absorbent surface to facilitate collection of the at least one fecal matter.

The top layer may further include a liquid blocking layer disposed on at least a portion of a bottom surface of the top layer opposite with respect to the absorbent surface to prevent movement of at least one of the at least one liquid and the at least one fecal matter therethrough.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates an isometric top view of a waste collection device, according to an exemplary embodiment of the present general inventive concept;

FIG. 2 illustrates an elevational bottom view of a top layer, according to an exemplary embodiment of the present general inventive concept; and

FIG. 3 illustrates an elevational bottom view of a bottom layer, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Waste Collection Device 100
Top Layer 110
Absorbent Surface 111
Reservoirs 112
Collection Channel 113
Liquid Blocking Layer 114
Inner Fasteners 115
Bottom Layer 120
Reinforced Body 121
Outer Fasteners 122

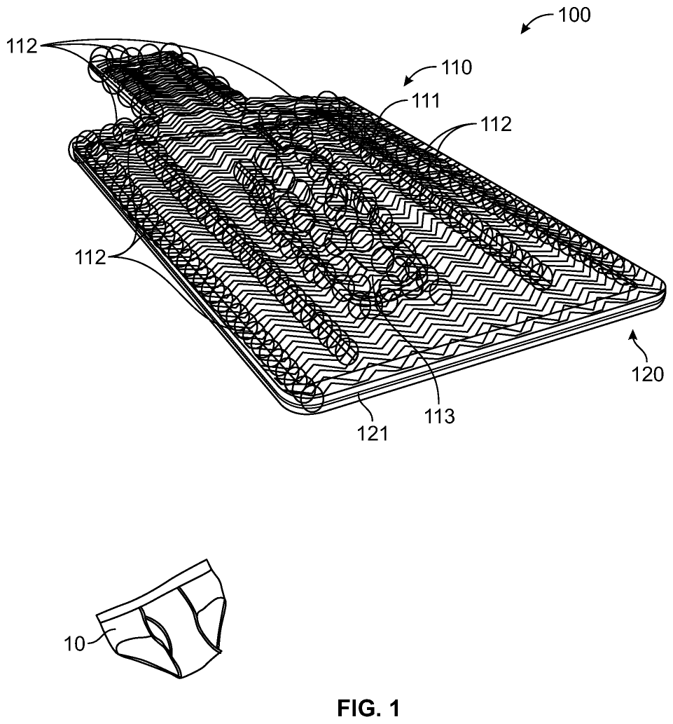

FIG. 1 illustrates an isometric top view of a waste collection device 100, according to an exemplary embodiment of the present general inventive concept.

The waste collection device 100 may be constructed from at least one of fabric, cloth, plastic, and rubber, etc., but is not limited thereto.

The waste collection device 100 may include a top layer 110 and a bottom layer 120, but is not limited thereto.

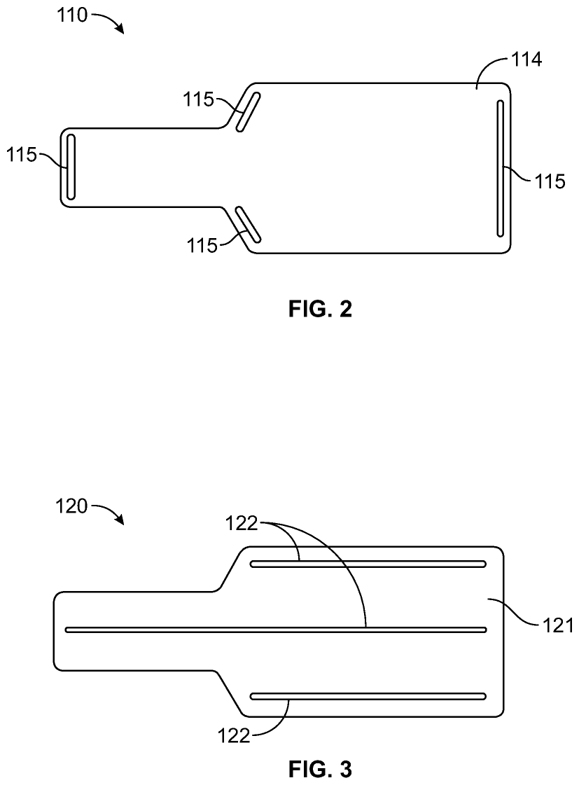

FIG. 2 illustrates an elevational bottom view of a top layer 110, according to an exemplary embodiment of the present general inventive concept.

The top layer 110 may include an absorbent surface 111, a plurality of reservoirs 112, a collection channel 113, a liquid blocking layer 114, and a plurality of inner fasteners 115, but is not limited thereto.

The absorbent surface 111 may be constructed of linen, cotton, rayon, wool, fleece, bamboo fabric, microfiber, plush, hemp, and flannel, but is not limited thereto. The absorbent surface 111 may be disposed on at least a portion of a top surface of the top layer 110. Optionally, the absorbent surface 111 may be disposed on an entirety of the top surface of the top layer 110. Referring to FIG. 1, the absorbent surface 111 may have a zig zag pattern to increase a volume of absorbent material in a smaller area. In other words, the absorbent surface 111 may have the zig zag pattern to compress an amount of the absorbent material on the top layer 110. As such, the absorbent surface 111 may absorb at least one liquid thereon. For example, the absorbent surface 111 may absorb urine and/or fecal matter thereon.

The plurality of reservoirs 112 may be disposed on and/or within at least a portion of the absorbent surface 111. More specifically, each of the plurality of reservoirs 112 may form a groove that is recessed with respect to a surrounding portion of the absorbent surface 111. In other words, each of the plurality of reservoirs 112 may have a lower elevation with respect to the absorbent surface 111. Therefore, each of the plurality of reservoirs 112 may receive and/or collect the at least one liquid therein in response to the at least one liquid moving (i.e., flowing) into at least one of the plurality of reservoirs 112.

The collection channel 113 may be longitudinally disposed along at least a portion of a length of the absorbent surface 111. Moreover, the collection channel 113 may be recessed with respect to a surrounding portion of the absorbent surface 111. Additionally, the collection channel 113 may be recessed with respect to each of the plurality of reservoirs 112. In other words, the collection channel 113 may have a lower elevation with respect to each of the plurality of reservoirs 112. The collection channel 113 may collect and/or store at least one fecal matter therein. Optionally, the collection channel 113 may be constructed to have less volume and/or density of absorbent material with respect to the surrounding portion of the absorbent surface 111 to facilitate reception and/or collection of the at least one fecal matter.

The liquid blocking layer 114 may be constructed of rubber, polyvinyl chloride (PVC), polyurethane, silicone elastomer, fluoropolymers, and wax, but is not limited thereto.

The liquid blocking layer 114 may be disposed on at least a portion of a bottom surface of the top layer 110 opposite with respect to the absorbent surface 111. The liquid blocking layer 114 may prevent movement of the at least one liquid and/or the at least one fecal matter therethrough. As such, the liquid blocking layer 114 may prevent damage (e.g., staining) to an undergarment 10 (e.g., briefs, boxers, panties) and/or other clothing from the at least one liquid and/or the at least one fecal matter.

Each of the plurality of inner fasteners 115 may include an adhesive, such as tape or glue, but is not limited thereto.

The plurality of inner fasteners 115 may be disposed on at least a portion of the liquid blocking layer 114.

FIG. 3 illustrates an elevational bottom view of a bottom layer 120, according to an exemplary embodiment of the present general inventive concept.

The bottom layer 120 may include a reinforced body 121 and a plurality of outer fasteners 122, but is not limited thereto.

The reinforced body 121 may be constructed of rubber, polyvinyl chloride (PVC), polyurethane, silicone elastomer, fluoropolymers, and wax, but is not limited thereto.

The reinforced body 121 may be more rigid with respect to the top layer 110. Additionally, the reinforced body 121 may prevent movement of the at least one liquid and/or the at least one fecal matter therethrough. In other words, the reinforced body 121 may be a second line of defense, such that the reinforced body 121 reinforces the liquid blocking layer 114 and prevents leaking of waste.

It is important to note that the plurality of fasteners 115 may removably connect to at least a portion of a top surface of the reinforced body 121. Also, the top layer 110 may be removed from the bottom layer 120 and exchanged for another top layer 110.

Each of the plurality of outer fasteners 122 may include an adhesive, such as tape or glue, but is not limited thereto.

The plurality of outer fasteners 122 may be disposed on at least a portion of a bottom surface of the reinforced body 121 opposite with respect to the top surface of the reinforced body 121. The plurality of outer fasteners 122 may removably connect to at least a portion of the undergarment 10.

Therefore, the waste collection device 100 may be connected to an interior of the undergarment 10 to protect the undergarment 10 and/or a user from contamination due to an unplanned discharge of waste.

The present general inventive concept may include a waste collection device 100 to connect to an interior of an undergarment 10, the waste collection device 100 including a top layer 110, including an absorbent surface 111 to absorb at least one liquid thereon, a plurality of reservoirs 112 disposed within at least a portion of the absorbent surface 111 to collect the at least one liquid therein, and a collection channel 113 longitudinally disposed along at least a portion of the absorbent surface 111 to collect at least one fecal matter therein, and a bottom layer 120 removably connected to at least a portion of the top layer 110 to prevent movement of at least one of the at least one liquid and the at least one fecal matter therethrough.

The absorbent surface 111 may have a zig zag pattern to increase a volume of absorbent material disposed thereon.

Each of the plurality of reservoirs 112 may form a groove that is recessed with respect to a surrounding portion of the absorbent surface 111.

The collection channel 113 may have less volume of absorbent material with respect to a surrounding portion of the absorbent surface 111 to facilitate collection of the at least one fecal matter.

The top layer 110 may further include a liquid blocking layer 114 disposed on at least a portion of a bottom surface of the top layer 110 opposite with respect to the absorbent surface 111 to prevent movement of at least one of the at least one liquid and the at least one fecal matter therethrough.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A waste collection device to connect to an interior of an undergarment, the waste collection device comprising:
a top layer, comprising:
an absorbent surface to absorb at least one liquid thereon,
a plurality of reservoirs disposed within at least a portion of the absorbent surface to collect the at least one liquid therein, and
a collection channel longitudinally disposed along at least a portion of the absorbent surface to collect at least one fecal matter therein; and
a bottom layer removably connected to at least a portion of the top layer to prevent movement of at least one of the at least one liquid and the at least one fecal matter therethrough.

2. The waste collection device of claim 1, wherein the absorbent surface has a zig zag pattern to increase a volume of absorbent material disposed thereon.

3. The waste collection device of claim 1, wherein each of the plurality of reservoirs forms a groove that is recessed with respect to a surrounding portion of the absorbent surface.

4. The waste collection device of claim 1, wherein the collection channel has less volume of absorbent material with respect to a surrounding portion of the absorbent surface to facilitate collection of the at least one fecal matter.

5. The waste collection device of claim 1, wherein the top layer further comprises:
a liquid blocking layer disposed on at least a portion of a bottom surface of the top layer opposite with respect to the absorbent surface to prevent movement of at least one of the at least one liquid and the at least one fecal matter therethrough.
